(19) United States
(12) Patent Application Publication
(10) Pub. No.: US 2023/0333042 A1
(43) Pub. Date: Oct. 19, 2023

(54) DEVICES AND METHODS ENABLING THE IMPROVED DETECTION OF ANALYTES BY MEANS OF REDUCED BACKGROUND SIGNAL ATTRIBUTABLE TO UNDESIRABLE REDOX-ACTIVE SPECIES IN A SAMPLE

(71) Applicant: University Of Cincinnati, Cincinnati, OH (US)

(72) Inventor: Jason Charles Heikenfeld, Cincinnati, OH (US)

(21) Appl. No.: 18/027,389

(22) PCT Filed: Sep. 24, 2021
(86) PCT No.: PCT/US2021/051949

§ 371 Date: Mar. 21, 2023

Related U.S. Application Data
(63) Continuation of application No. 63/150,931, filed on Feb. 18, 2021.
(60) Provisional application No. 63/085,469, filed on Sep. 30, 2020.

(51) Int. Cl. G01N 27/327; C12Q 1/00; G01N 27/30

(52) U.S. Cl. CPC G01N 27/3271; C12Q 1/006; G01N 27/308; G01N 27/3277

(57) ABSTRACT

A device 100 for sensing the presence of at least one analyte in a sample 190 includes a sensor 120 for detecting the presence of at least one redox-active species and a component for reducing the concentration of redox-active species in the sample. A method for detecting the presence of at least one redox-inactive analyte in the sample includes reducing the concentration of redox-active species in the sample, wherein the at least one analyte then interacts with an element 122 in a manner resulting in the detection of a redox-active species attributable to the analyte. This method and device enable the improved detection of analytes with concentration equal to or lower than 1 mM or of analytes with concentrations equal to or lower than 1 μM by reducing the background signal of redox-active species in the sample not attributable to the analyte.

### CROSS-REFERENCE TO RELATED APPLICATIONS

This application claims priority to, and the benefit of the filing date of, U.S. Provisional Application No. 63/085,469 filed Sep. 30, 2020 and U.S. Provisional Application No. 63/150,931 filed Feb. 18, 2021, the disclosures of which are incorporated by reference herein in their entireties.

### TECHNICAL FIELD

The present invention relates to the use of electrochemical sensors for the detection of an analyte or analytes in a sample

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical sensors are useful for detecting analytes capable of either undergoing a redox reaction with an electrode or otherwise able to increase the signal generated by the sensor. Electrochemical sensors detect the presence of redox-active species through the current generated by a redox reaction occurring at the electrode(s) of the sensor. One embodiment of electrochemical sensors is the enzymatic electrochemical sensor. The enzymatic electrochemical sensor relies on an enzymatic reaction with an analyte that directly or indirectly produces a redox-active species that can be detected using electrical, optical, mechanical, or other suitable measurement means. In these enzymatic electrochemical sensors, the enzyme acts as an intermediary while the electrode measures the redox-active species. This powerful sensing system has driven the commercial success of widely used ethanol, glucose, and lactate electrochemical sensors. Another useful embodiment of electrochemical sensors is the electrochemical aptamer sensor. Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox-active molecule (redox couple) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of concentration of the analyte.

Recent research efforts have been directed to developing electrochemical sensors for highly dilute analytes which are typically found in μM concentrations or lower, such as cortisol. Even analytes which are already commonly detected with these systems (e.g., ethanol detection using breathalyzers) may benefit from an improved ability to detect μM or lower concentrations of the analyte in different situations or for different applications. However, the challenge with using electrochemical sensors for detecting many of these highly dilute analytes is the presence of redox-active species in the sample fluid other than those attributable to the analyte that would otherwise generate the same sensor signal generated by the detection of the target analyte. These other redox-active species which generate the same sensor signal create a background signal which inhibits the ability to detect small analyte concentrations. For example, blood contains numerous other solutes that are both (1) redox-active and (2) give rise to a background current that limits the detection of a highly dilute analyte, such as cortisol. Therefore, what is needed are devices and methods to reduce background current due to redox-active solutes present in a sample fluid, such that detection devices can be used to detect highly dilute analytes.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Embodiments of the disclosed invention are directed to devices and methods for enabling the improved detection of analytes by reducing background current caused by redox-active solutes in a sample fluid.

And so, one aspect of the present invention is directed to a device for detecting at least one analyte in a sample fluid. The device includes (1) a sensor for detecting an analyte-related redox-active species in a sample fluid, and (2) at least one depleting component that reduces the concentration of redox-active species in the sample fluid that are unrelated to the at least one analyte. The sensor may also include an element or component that interacts with any analyte in sample fluid to generate the analyte-related redox-active species. Alternatively, the element or component may itself include, or contain, the analyte-related redox active species. And the sensor may include an electrode, wherein the analyte-related redox-active species interacts with the electrode in a manner that allows the presence or amount of analyte-related redox-active species to be determined.

Another aspect of the present invention is directed to a method for detecting at least one analyte in a sample fluid. The method includes reducing the concentration of redox-active species in a sample fluid that are unrelated to the at least one analyte. The method also includes causing at least one analyte in a sample fluid to interact with an element that (1) produces at least one analyte-related redox-active species in the presence of the at least one analyte, (2) contains at least one analyte-related redox-active species which is made more detectable through interaction with the at least one analyte, or (3) otherwise enables the detection of at least one analyte-related redox-active species attributable to the presence of the at least one analyte. And the method also includes detecting a presence or measuring an amount of the at least one analyte-related redox-active species attributable to the interaction between the at least one analyte and the element.

Another aspect of the present invention is directed to embodiments for the depleting component. In one such embodiment, the depleting component contains a first electrode and a second electrode. The second electrode may be configured to be permeable to at least one diffusant including, for example, a sample fluid, at least one analyte, and undesirable redox-active species. The second electrode may further include a first end, a second end opposite the first end, and at least one diffusion pathway between the first end and second end. The at least one diffusion pathway may be configured to have a shortest diffusion pathway length and an average maximum second electrode surface distance. The difference between the shortest diffusion pathway length and the average maximum second electrode surface distance may be partially responsible for a significant reduction in an initial concentration of an undesirable redox-active species. The difference between the shortest diffusion pathway length and the average maximum second electrode surface distance may improve a limit of detection of at least one analyte.

In aspects of the various device and method embodiments of the invention, analyte in the sample fluid specifically causes the presence or increased detection of an analyte-related redox-active species that did not exist in the sample fluid prior to contact with the device. In addition, the depleting component reduces the presence of undesirable redox-active species other than the analyte-related redox-active species. This reduces background current due to redox-active solutes present in a sample fluid, such that detection devices can be used to detect highly dilute analytes.

### BRIEF DESCRIPTION OF THE DRAWINGS

The objects and advantages of the disclosed invention will be further appreciated in light of the following detailed descriptions and drawings in which:

FIG. 1 is a cross-sectional view of a device according to an embodiment of the disclosed invention.

FIG. 2 is a cross-sectional view of a device according to another embodiment of the disclosed invention.

FIG. 3 is a cross-sectional view of a device according to yet another embodiment of the disclosed invention.

### DEFINITIONS

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

The devices and methods described herein encompass the use of sensors. As used herein, the term “sensor” means any device that relies the presence of a redox-active species to detect or determine the concentration of a target analyte in a sample fluid. Such sensors include enzymatic sensors that interact with the analyte to produce redox-active species. Such sensors also include aptamer sensors which have an attached redox couple which provides the signal transduction mechanism. The specific implementation of such sensors does not limit the present invention, which aims to instead resolve the issue of redox-active species in the sample fluid.

As used herein, the term “redox-active species” means any composition of matter that is capable of undergoing a redox reaction with the sensor to generate a sensor signal. As used herein, the term “undesirable redox-active species” means any redox-active species in a sample that would generate a sensor signal similar to the signal generated by the presence of the target analyte but whose detection would not be attributable to or increased by the presence of the target analyte. As used herein, the term “depleting component” means any component that is capable of reducing the concentration of any undesirable redox-active species present in a sample.

As used herein, the term “analyte” means any composition of matter that the sensor is designed or intended to detect, either directly or indirectly, for example, including an inorganic molecule, an organic molecule, a small molecule drug, a metabolite, a hormone, a peptide, a protein, a carbohydrate, a nucleic acid, a chemical, a particle, or any other composition of matter. A drug analyte may be of any type, for example, including drugs for the treatment of cardiac system, the treatment of the central nervous system, that modulate the immune system, that modulate the endocrine system, an antibiotic agent, a chemotherapeutic drug, or an illicit drug. The analyte may comprise a naturally-occurring factor, for example a hormone, metabolite, growth factor, neurotransmitter, etc. The target analyte may comprise any other species of interest, for example, species such as pathogens (including pathogen induced or derived factors), nutrients, and pollutants, etc.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and, for purposes of brevity and focus on inventive aspects, such components may not be explicitly shown in the diagrams or described in the embodiments of the disclosed invention. All ranges of parameters disclosed herein include the endpoints of the ranges.

As described above, one aspect of the present invention is directed to a device for detecting at least one analyte in a sample fluid. The device includes (1) a sensor for detecting an analyte-related redox-active species in a sample fluid, and (2) at least one depleting component that reduces the concentration of redox-active species in the sample fluid that are unrelated to the at least one analyte. The sensor may also include an element or component that interacts (or includes a component that interacts) with any analyte in sample fluid to generate the analyte-related redox-active species. And the sensor may include an electrode, wherein the analyte-related redox-active species interacts with the electrode in a manner that allows the presence or amount of analyte-related redox-active species to be determined.

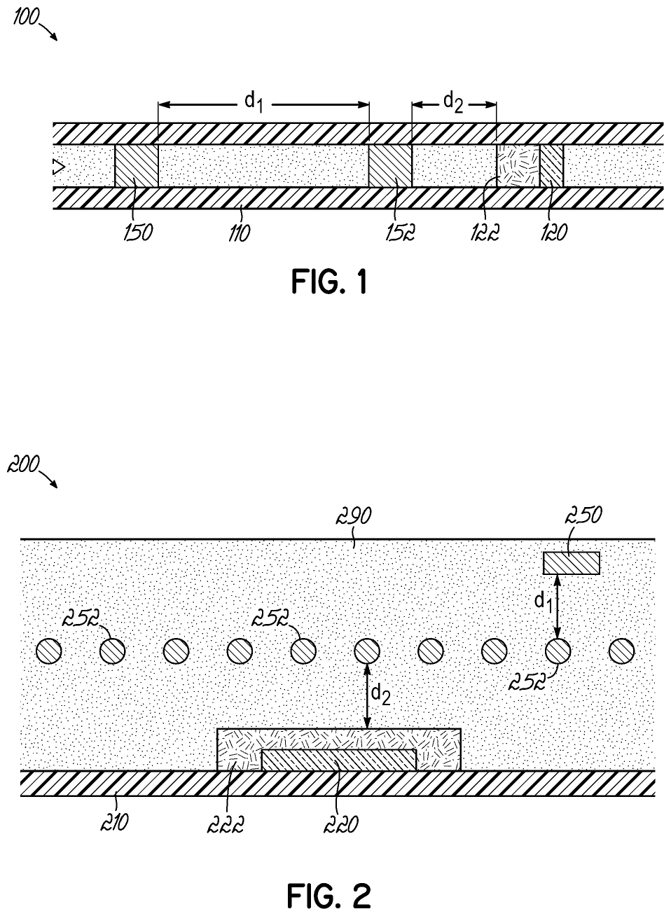

With reference to FIG. 1, in one embodiment of the disclosed invention, a device 100 includes at least one substrate 110 which can support, be exposed to, or transport a sample fluid 190. This sample fluid may contain an at least one analyte to be measured. In various embodiments, the substrate 110 may include a material such as polyethylene terephthalate (PET) or glass. The device 100 further includes at least one depleting component, which in FIG. 1 is represented by first and second electrodes 150, 152. Non-limiting examples of materials that may be used for first and second electrodes 150, 152 include a metal mesh, porous carbon, or a conventional electrode. In one embodiment, first electrode 150 is a cathode and second electrode 152 is an anode. In another embodiment, first electrode 150 is an anode and second electrode 152 is a cathode.

In various embodiments, the first electrode 150 and the second electrode 152 are separated by a distance d₁. In certain embodiments, d₁ is equal to the shortest distance between first electrode 150 and second electrode 152. In one embodiment, first electrode 150 (being a cathode electrode) and second electrode 152 (being an anode electrode) are separated by a distance d₁ that aids in allowing reduction of redox-active species that are unrelated to the analyte (i.e., undesirable redox-active species) at first electrode 150 and oxidation of undesirable redox-active species at second electrode 152. In such an embodiment (where first electrode 150 is a cathode and second electrode 152 is an anode), d₁ is configured to be a sufficient distance (μm's, mm's, or cm's depending on time-scale and geometry) such that a concentration of reduced species inside or near second electrode 152, as compared to an area inside or near first electrode 150, will be significantly diminished. In other words, as sample fluid 190 is introduced to device 100, it may contain any number of redox-active species, some of which are oxidizable and some of which are reducible (some of which, then could create background current that would negatively impact the accuracy or operation of the sensor device). As sample fluid 190 proceeds through device 100 (in this embodiment, and from left to right as shown in FIG. 1), it will first encounter the cathode (first electrode 150). As the redox-active species in sample fluid approach the cathode, reducible species in the sample fluid will be reduced, while oxidizable species will not be significantly affected. Thus, as the sample fluid 190 gets closer to the cathode, the concentration of reduced species will increase and a substantial majority, or nearly all, or all reducible species in the sample fluid 190 will have been reduced. And so, sample fluid 190 that progresses past the cathode into a portion of device 100 between first electrode 150 and second electrode 152 having distance d₁ will be laden with reduced species (including both those originally reduced and those reduced by cathode). As those reduced species progress from the cathode (first electrode 150) to the anode (second electrode 152) over distance d₁, the reduced species will begin to oxidize as they approach the anode—and, at the anode, a substantial majority, or nearly all, or all of the reduced species will have been oxidized. In this oxidized state, the redox-active species no longer provide background current that would disrupt operation of device in detecting or measuring the target analyte. Distance, d₁, can be calculated to have a length sufficient to result in a substantial majority, or nearly all, or all of the reduced species being oxidized as they pass the anode (second electrode 152) and progress toward an element 122.

In another embodiment, first electrode 150 (being an anode electrode) and second electrode 152 (being a cathode electrode) are separated by a distance d₁ that aids in allowing oxidation of undesirable redox-active species at first electrode 150 and reduction of undesirable redox-active species at second electrode 152. In such an embodiment (where first electrode 150 is an anode and second electrode 152 is a cathode), d₁ is configured to be a sufficient distance (μm's, mm's, or cm's depending on time-scale and geometry) such that a concentration of oxidized species inside or near second electrode 152, as compared to an area inside or near first electrode 150, will be significantly diminished. In other words, as sample fluid 190 is introduced to device 100, it may contain any number of redox-active species, some of which are oxidizable and some of which are reducible (some of which, then could create background current that would negatively impact the accuracy or operation of the sensor device). As sample fluid 190 proceeds through device 100 (in this embodiment, and from left to right as shown in FIG. 1), it will first encounter the anode (first electrode 150). As the redox-active species in sample fluid approach the anode, oxidizable species in the sample fluid will be oxidized, while reducible species will not be significantly affected. Thus, as the sample fluid 190 gets closer to the anode, the concentration of oxidized species will increase and a substantial majority, or nearly all, or all oxidizable species in the sample fluid 190 will have been oxidized. And so, sample fluid 190 that progresses past the anode into a portion of device 100 between first electrode 150 and second electrode 152 having distance d₁ will be laden with oxidized species (including both those originally oxidized and those oxidized by anode). As those oxidized species progress from the anode (first electrode 150) to the cathode (second electrode 152) over distance d₁, the oxidized species will begin to reduce as they approach the cathode—and, at the cathode, a substantial majority, or nearly all, or all of the oxidized species will have been reduced. In this reduced state, the redox-active species no longer provide background current that would disrupt operation of device in detecting or measuring the target analyte. Distance, d₁, can be calculated to have a length sufficient to result in a substantial majority, or nearly all, or all of the oxidized species being reduced as they pass the cathode (second electrode 152) and progress toward element 122. In embodiments where first electrode 150 is an anode and second electrode 152 is a cathode, d₁ is configured to be a sufficient distance (μm's, mm's, or cm's depending on time-scale and geometry) such that a concentration of oxidized species inside or near second electrode 152, as compared to an area inside or near first electrode 150, will be significantly diminished.

The device 100 of the embodiment of FIG. 1 further includes element 122 that contains or produces a redox-active species that is specific to the analyte to be detected (that redox-active species referred to herein as an “analyte-related redox-active species”). Element 122 is separated from second electrode 152 by a distance d₂. In one embodiment, the device 100 further includes a third electrode 120. This third electrode 120 may be proximal, in certain embodiments, to element 122 (e.g., as shown in FIG. 1). In one embodiment, the third electrode 120 may comprise an anode. In an alternate embodiment, the third electrode 120 may comprise a cathode. In various embodiments, the third electrode 120 can be considered to be an electrochemical sensor. In alternate embodiments, a combination of the third electrode 120 and the element 122 can be considered to be an electrochemical sensor.

In various embodiments, element 122 may take different forms or include different components. As described above, in certain embodiments, element 122 may be a component or material that produces an analyte-related redox-active species. In an example of such an embodiment, the element 122 comprises at least one enzyme which produces an analyte-related redox-active species. Thus, for example, the device 100 may be one that identifies the presence of, or measures the amount of, glucose in a sample fluid (thus, glucose is the analyte). Enzymes can be developed along with chemistries for sensing of glucose, lactate, ethanol, cortisol, and other analytes. In this example, the third electrode 120 may be an anode such as platinum and the element 122 may comprise an enzyme such as glucose oxidase, which is separated from second electrode 152 by distance d₂. In this embodiment, third electrode 120 functions as a sensor and will not substantially oxidize undesirable redox-active species contained in sample fluid 190 because such undesirable redox-active species have already been substantially oxidized by effect of first and second electrodes 150 and 152 as the sample fluid 190 proceeds along device 100 in the direction shown by arrow in FIG. 1. As a result of this reduction of the concentration of the undesirable redox-active species in sample fluid 190 prior to the sample fluid reaching element 122, the glucose analyte can react with element 122 (being the enzyme in this example) to generate peroxide or other species (i.e., to produce the analyte-related redox-active species) that is then oxidized at third electrode 120 and measured as an electrical current with minimized background interference. Further, due to this reduction of background interference as electrode 152 has oxidized most or all other oxidizable species, the limit of detection of the glucose analyte can be improved significantly.

In one specific embodiment, the limit detection of the glucose analyte can be further improved if third electrode 120 is comprised of a material such as boron-doped diamond to further reduce background current in water.

In an alternate embodiment, element 122 may contain, or may itself be, an analyte-related redox-active species. In such an embodiment, the element 122 may comprise at least one redox-tagged aptamer that is capable of binding specifically to the analyte. And so, for example, the device 100 may be one that identifies the presence of, or measures the amount of, cortisol in a sample fluid (thus, cortisol is the analyte). The third electrode 120 may comprise any suitable electrode material for electrochemical sensing, including, for example: gold or any gold-coated metal or material, titanium, tungsten, platinum, carbon, aluminum, copper, palladium, mercury films, silver, oxide-coated metals, semiconductors, graphite, carbon nanotubes, and any other conductive material upon which biomolecules can be conjugated. The element 122 may comprise a redox-tagged aptamer, such as aptamer including a redox-reporter of methylene blue, which is separated from second electrode 152 by distance d₂. In this example, third electrode 120 functions as a sensor and will not substantially oxidize undesirable redox-active species contained in sample fluid 190 because such undesirable redox-active species have already been substantially reduced and oxidized by effect of first and second electrodes 150 and 152. When the cortisol analyte binds to the redox-tagged aptamer element 122, the aptamer changes structure and alters the proximity of the redox-reporter to the third electrode 120. As a result of reducing the concentration of the undesirable redox-active species in sample fluid 190 prior to the sample fluid interacting with element 122, the signal generated by the analyte-related redox-active species aptamer bound to analyte is measured as an electrical current with minimized background interference. As a result, the limit of detection of the analyte can be improved significantly. In a further embodiment, the limit detection of the analyte can be further improved if third electrode 120 is comprised of a material such as boron-doped diamond to further reduce background current in water.

Additionally, in one embodiment, all electrodes (first, second, and third electrodes 150, 152, 120) are used simultaneously. In other embodiments, not all of first, second, and third electrodes 150, 152, 120 are used simultaneously. Their representations or polarities as shown in FIG. 1 are non-limiting examples only and may be changed based on factors such as type of sensor and type of species to be detected. In one embodiment, first and second electrodes 150 and 152 are used continuously or pulsed electrically, thereby depleting sample fluid 190 of oxidizable species as it flows toward third electrode 120. In an alternate embodiment, first electrode 150 is constantly run with positive voltage, and second and third electrodes 152 and 120 are alternately electrically floating or supplied with negative voltage, such that both second and third electrodes 152, 120 deplete undesirable redox-active species in the sample fluid 190. Then, when it is time for performing a sensing measurement, third electrode 120 is kept floating or at a suitable voltage such that, for seconds or minutes, element 122 has adequate time to generate a measurable amount of analyte-related redox-active species. In one embodiment, this is done to allow enzymatically created oxidizable byproducts to be produced. In yet another embodiment, this is done to allow enzymatically created reducible byproducts to be produced. In another embodiment, third electrode 120 is kept floating for seconds or minutes to allow the change in aptamer structure to increase the altered distance between the redox-reporter and the third electrode 120, and a voltage is applied to third electrode 120 to electrically measure aptamer such as a chronoamperometric measurement, which will show a slower chronoamperometric response the further the redox-reporter is away from the electrode.

In one embodiment, to ensure that undesirable redox-active species are kept substantially away from sensor 120, preferably d₁ is greater than d₂. In a further embodiment, preferably d₁ is at least ten times greater than d₂. In another further embodiment, preferably d₁ is at least one hundred times greater than d₂. In a yet further embodiment, preferably d₁ is at least one thousand times greater than d₂. In an alternate embodiment, as will be taught later, d₁ and d₂ can also be similar in size. In a further embodiment, d₁ and d₂ can be similar in size because the second electrode 152 is constructed in a manner such that it is difficult for the undesirable redox-active species to diffuse through the second electrode 152 without being reduced or oxidized by the second electrode 152. In various embodiments, to aid in the reduction of undesirable redox-active species, a flow of sample fluid 190 is implemented where the sample fluid flows in the direction from first electrode 150 toward the second electrode 152, element 122, and third electrode 120. This flow may be implemented by pumping, capillary, or other fluid flow inducing means.

Another embodiment in accordance with principles of the present invention is shown in FIG. 2. Here, a device 200 includes at least one substrate 210 which can support, be exposed to, or transport a sample fluid 290. The sample fluid 290 may contain an analyte to be measured. In various embodiments, the substrate 210 comprises materials such as PET or glass. The device 200 further includes at least one depleting component, which in FIG. 2 is represented by an at least one first electrode 250 and an at least one second electrode 252. Non-limiting examples of first and second electrodes 250, 252 include a metal mesh, porous carbon, or a conventional electrode. In various embodiments, first electrode 250 may include using one electrode or more than one electrode. In various embodiments, second electrode 252 may include using one electrode or more than one electrode. In one embodiment, the at least one first electrode 250 is a cathode and the at least one second electrode 252 is an anode. In another embodiment, the at least one first electrode 250 is an anode and the at least one second electrode 252 is a cathode.

In various embodiments, each of the at least one electrode 250 is separated from each of the at least one electrode 252 by a distance. In various embodiments, a distance d₁ is the shortest distance between a closest pair of the at least one first electrode 250 and the at least second electrode 252. In one embodiment, the at least one first electrode 250 (being a cathode) and the at least one second electrode 252 (being an anode) are separated by a distance d₁ that aids in allowing reduction of undesirable redox-active species at the at least one first electrode 250 and oxidation of undesirable redox-active species at the at least one second electrode 252. In such an embodiment (where the at least one first electrode 250 is a cathode and the at least one second electrode 252 is an anode), d₁ is configured to be a sufficient distance (μm's, mm's, or cm's depending on time-scale and geometry) such that a concentration of reduced species inside or near the at least one second electrode 252, as compared to an area inside or near the at least one first electrode 250, will be significantly diminished. In other words, as sample fluid 290 is introduced to device 200, it may contain any number of redox-active species, some of which are oxidizable and some of which are reducible (some of which, then could create background current that would negatively impact the accuracy or operation of the sensor device). As sample fluid 290 proceeds through device 200 (in this embodiment, and from top to bottom as shown in FIG. 2), it will first encounter the at least one cathode (first electrode 250). As the redox-active species in sample fluid approach the at least one cathode, reducible species in the sample fluid will be reduced, while oxidizable species will not be significantly affected. Thus, as the sample fluid 290 gets closer to the at least one cathode, the concentration of reduced species will increase and a substantial majority, or nearly all, or all reducible species in the sample fluid 290 will have been reduced. And so, sample fluid 290 that progresses past the at least one cathode into a portion of device 200 between the at least one first electrode 250 and the at least one second electrode 252 having distance d₁ will be laden with reduced species (including both those originally reduced and those reduced by the at least one cathode). As those reduced species progress from the at least one cathode (first electrode 250) to the at least one anode (second electrode 252) over a distance greater than or equal to d₁, the reduced species will begin to oxidize as they approach the at least one anode—and, at the at least one anode, a substantial majority, or nearly all, or all of the reduced species will have been oxidized. In this oxidized state, the redox-active species no longer provide background current that would disrupt operation of device in detecting or measuring the target analyte. Distance, d₁, can be calculated to have a length sufficient to result in a substantial majority, or nearly all, or all of the reduced species being oxidized as they pass the at least one anode (second electrode 252) and progress toward an element 222. In embodiments where the at least one first electrode 250 is a cathode and the at least one second electrode 252 is an anode, d₁ is configured to be a sufficient in distance (μm's, mm's, or cm's depending on time-scale and geometry) such that a concentration of reduced species inside or near second electrode 252, as compared to an area inside or near first electrode 250, will be significantly diminished.

In one embodiment, the at least one first electrode 250 (being an anode) and the at least one second electrode 252 (being a cathode) are separated by a distance d₁ that aids in allowing oxidation of undesirable redox-active species at the at least one first electrode 250 and reduction of undesirable redox-active species at the at least one second electrode 252. In such an embodiment (where the at least one first electrode 250 is an anode and the at least one second electrode 252 is a cathode), d₁ is configured to be a sufficient distance (μm's, mm's, or cm's depending on time-scale and geometry) such that a concentration of oxidized species inside or near the at least one second electrode 252, as compared to an area inside or near the at least one first electrode 250, will be significantly diminished. In other words, as sample fluid 290 is introduced to device 200, it may contain any number of redox-active species, some of which are reducible and some of which are oxidizable (some of which, then could create background current that would negatively impact the accuracy or operation of the sensor device). As sample fluid 290 proceeds through device 200 (in this embodiment, and from top to bottom as shown in FIG. 2), it will first encounter the at least one anode (first electrode 250). As the redox-active species in sample fluid approach the at least one anode, ozidizable species in the sample fluid will be oxidized, while reducible species will not be significantly affected. Thus, as the sample fluid 290 gets closer to the at least one anode, the concentration of oxidized species will increase and a substantial majority, or nearly all, or all oxidizable species in the sample fluid 290 will have been oxidized. And so, sample fluid 290 that progresses past the at least one anode into a portion of device 200 between the at least one first electrode 250 and the at least one second electrode 252 having distance d₁ will be laden with oxidized species (including both those originally oxidized and those oxidized by the at least one anode). As those oxidized species progress from the at least one anode (first electrode 250) to the at least one cathode (second electrode 252) over a distance greater than or equal to d₁, the oxidized species will begin to reduce as they approach the at least one cathode—and, at the at least one cathode, a substantial majority, or nearly all, or all of the oxidized species will have been reduced. In this reduced state, the redox-active species no longer provide background current that would disrupt operation of device in detecting or measuring the target analyte. Distance, d₁, can be calculated to have a length sufficient to result in a substantial majority, or nearly all, or all of the oxidized species being reduced as they pass the at least one cathode (second electrode 252) and progress toward element 222. In embodiments where the at least one first electrode 250 is an anode and the at least one second electrode 252 is a cathode, d₁ is configured to be a sufficient in distance (μm's, mm's, or cm's depending on time-scale and geometry) such that a concentration of oxidized species inside or near second electrode 252, as compared to an area inside or near first electrode 250, will be significantly diminished.

Still referring to FIG. 2, the device 200 may further include element 222 that contains or produces an analyte-related redox-active species. Element 222 is separated from each of the at least one second electrode by a distance. In various embodiments, a distance d₂ is a shortest distance between the element 222 and the at least one second electrode 252 closest to element 222. In various embodiments, the device 200 further includes a third electrode 220. This third electrode 220 may be proximal, in certain embodiments, to element 222 (e.g., as shown in FIG. 2). In one embodiment, the third electrode 220 may comprise a cathode. In an alternate embodiment, the third electrode may comprise an anode. In various embodiments, the third electrode 220 can be considered to be an electrochemical sensor. In alternate embodiments, a combination of the third electrode 220 and the element 222 can be considered to be an electrochemical sensor.

In various embodiments, element 222 may take different forms or include different components. As described above, in certain embodiments, element 222 may be a component or material that produces an analyte-related redox-active species. In an example of such an embodiment, the element 222 comprises at least one enzyme which produces an analyte-related redox-active species. Thus, for example, the device 200 may be one that identifies the presence of, or measures the amount of, glucose in a sample fluid (thus, glucose is the analyte). In this example, third electrode 220 may be an anode and the element 222 may comprise an enzyme such as glucose oxidase, and element 222 may be separated from each of the at least one second electrode 252 by a distance greater than or equal to distance d₂. In this embodiment, third electrode 220 functions as a sensor and will not substantially oxidize undesirable redox-active species contained in sample fluid 290 because such undesirable redox-active species have already been substantially oxidized by effect of electrodes 250, 252. As a result of this reduction of the concentration of the undesirable redox-active species in sample fluid 290, the glucose analyte can react with element 222 (being the enzyme in this example) to generate peroxide (i.e., to produce the analyte-related redox-active species) that is then oxidized at electrode 220 and measured as an electrical current with minimized background interference. Further, due to this reduction of background interference, the limit of detection of the glucose analyte can be improved significantly.

In an alternate embodiment, element 222 may contain, or be, an analyte-related redox-active species. In such an embodiment, the element 222 may comprise at least one redox-tagged aptamer that is capable of binding specifically to the analyte. And so, for example, the device 200 may be one that identifies the presence of, or measures the amount of, cortisol in a sample fluid (thus, cortisol is the analyte). The third electrode 220 may comprise any suitable electrode material for electrochemical sensing, including, for example: gold or any gold-coated metal or material, titanium, tungsten, platinum, carbon, aluminum, copper, palladium, mercury films, silver, oxide-coated metals, semiconductors, graphite, carbon nanotubes, and any other conductive material upon which biomolecules can be conjugated. The element 222 may comprises a redox-tagged aptamer, such as aptamer including a redox-reporter of methylene blue, which is separated from the at least one second electrode 252 by a distance greater than or equal to distance d₂. In this example, electrode 220 functions as a sensor and will not substantially oxidize undesirable redox-active species contained in sample fluid 290 because such undesirable redox-active species have already been substantially reduced and oxidized by effect of electrodes 250 and 252. When the cortisol analyte binds to the redox-tagged aptamer element 222, the aptamer changes structure and alters the proximity of the redox-reporter to the electrode 220. As a result of reducing the concentration of the undesirable redox-active species in sample fluid 290, the signal generated by the analyte-related redox-active species aptamer bound to analyte is measured as an electrical current with minimized background interference. As a result, the limit of detection of the analyte can be improved significantly. In a further embodiment, the limit detection of the analyte can be further improved if electrode 220 is comprised of a material such as boron-doped diamond to further reduce background current in water.

With reference to FIG. 2, in one embodiment all electrodes 220, 250, 252 are used simultaneously. In other embodiments, not all electrodes 220, 250, 252 are used simultaneously. Their representations or polarities as shown in FIG. 2 are non-limiting examples only and may be changed based on factors such as type of sensor and type of species to be detected. In one embodiment, electrodes 250, 252 are used continuously or pulsed electrically, thereby depleting sample fluid 290 of undesirable redox-active species. In an alternate embodiment, the at least one electrode 250 is constantly run with positive (anodic) voltage, and electrodes 252, 220 are alternately electrically floating or supplied with negative (cathodic) voltage, such that both electrodes 252, 220 deplete undesirable redox-active species in the sample fluid 290. Then, when it is time for doing a sensing measurement, electrode 220 is kept floating or at a suitable voltage such that, for seconds or minutes, element 222 has adequate time to generate a measurable amount of analyte-related redox-active species. In one embodiment, this is done to allow enzymatically created oxidizable byproducts to be produced. In yet another embodiment, this is done to allow enzymatically created reducible byproducts to be produced. In another embodiment, electrode 220 is kept floating for seconds or minutes to allow a change in aptamer structure to alter the proximity between the redox-reporter and the electrode 220. Afterward, a voltage is applied to third electrode 220 to electrically measure aptamer such as a chronoamperometric measurement which will show a faster chronoamperometric response the closer the redox-reporter is to the electrode. In various embodiments, the sample fluid 290 may be caused to flow within the device 200.

In one embodiment, to ensure that undesirable redox-active species other than those produced or contained by element 222 are kept substantially away from sensor 220, preferably d₁ is greater than d₂. In a further embodiment, preferably d₁ is at least ten times greater than d₂. In another further embodiment, preferably d₁ is at least one hundred times greater than d₂. In another further embodiment, d₁ is at least one thousand times greater than d₂. In an alternate embodiment, as will be taught later, d₁ and d₂ can also be similar in size. In a further embodiment, d₁ and d₂ can be similar in size because the at least one second electrode 252 is constructed in a manner such that it is difficult for the undesirable redox-active species to diffuse through the at least one second electrode 252 without being reduced or oxidized by the at least one second electrode 252. In another embodiment, to aid in the reduction of undesirable redox-active species, a flow of sample fluid 290 is implemented where the sample fluid flows in the direction from the at least one first electrode 250 toward the at least one second electrode 252, element 222, and third electrode 220. In a further embodiment, this flow is implemented by pumping, capillary, or other fluid flow inducing means. In yet another embodiment, sample fluid 290 is interstitial fluid and the device 200 has been placed into the dermis of the skin.

While at least three electrodes are shown in the embodiments as particularly illustrated in FIG. 1 (electrodes 150, 152, and 120) and FIG. 2 (electrodes 250, 252, and 220), other possible embodiments of the present invention (as described above) encompass the use of a two-electrode system. A system with at least three electrodes may be desired in various instances, because the presence of the at least one additional electrode before the sensor 120, 220 further assists in removing most or all of the reducible or oxidizable species. This results in even lower background current that would otherwise stem from undesirable redox-active species present in the sample fluid 190, 290. However, there may be other instances where the two-electrode system may be desired.

Another aspect of the present invention is directed to a method for detecting at least one analyte in a sample fluid. The method includes reducing the concentration of redox-active species in a sample fluid that are unrelated to the at least one analyte. The method also includes causing at least one analyte in a sample fluid to interact with an element that (1) produces at least one analyte-related redox-active species in the presence of the at least one analyte, and/or (2) contains at least one analyte-related redox-active species which is made more detectable through interaction with the at least one analyte, or (3) otherwise enables the detection of at least one analyte-related redox-active species attributable to the presence of the at least one analyte. And the method also includes detecting a presence or measuring an amount of the at least one analyte-related redox-active species attributable to the interaction between the at least one analyte and the element.

In the method, the step of causing the at least one analyte to interact with the element may include causing the sample fluid to flow along a path, wherein that path includes: (1) at least one redox-active species depleting component that is capable of reducing the concentration of undesirable redox-active species in the sample fluid; (2) the element; and (3) a sensor for detecting the presence of the at least one redox-active species attributable to the presence of the analyte in the sample.

Further, detecting a presence or measuring an amount of the analyte-related redox-active species may further include measuring an initial electrical current between the at least one electrode and the at least one analyte-related redox-active species. And the method may yet further include detecting and/or measuring a change from the initial electrical current between the at least one electrode and the at least one analyte-related redox-active species following bringing the sample fluid into proximity with the electrode.

Another aspect of the present invention is directed to additional embodiments of second electrode 152 or 252. Previously mentioned examples of second electrode 152, 252 include a mesh, metal foam, or other suitable species. In one such embodiment, the second electrode 152, 252 is configured to be permeable to at least one diffusant including sample fluid 190, 290, the analyte, and undesirable redox-active species. In various embodiments, second electrode 152, 252 is configured relative to the third electrode 120, 220 such that undesirable redox-active species diffuse through the second electrode 152, 252 before interacting with the third electrode 120, 220. In one such embodiment, undesirable redox-active species diffuse through the second electrode 152, 252 before interacting with the third electrode 120, 220 because the second electrode is placed on top of the sensor.

Thus, in certain embodiments, the second electrode 152, 252 includes a first end of the second electrode, a second end of the second electrode located opposite of the first end of the second electrode, and at least one diffusion pathway. The first end of the second electrode includes at least one point configured to allow entry of the at least one diffusant. The second end of the second electrode includes at least one point configured to allow departure of the at least one diffusant. The at least one diffusion pathway includes at least one connected pathway, a shortest diffusion pathway length, and an average maximum second electrode surface remoteness. The at least one connected pathway includes a first end of the connected pathway and a second end of the connected pathway. The first end of the connected pathway is located on the first end of the second electrode and includes the at least one point configured to allow entry of the at least one diffusant. The second end of the connected pathway is located on the second end of the second electrode and includes the point configured to allow departure of the at least one diffusant. In further regard to the various embodiments, the connected pathway is configured such that the point configured to allow entry of the at least one diffusant and the point configured to allow departure of the at least one diffusant are in fluid communication. The shortest diffusion pathway length spans a distance d₃ along a shortest path from the first end of the connected pathway to the second end of the connected pathway. The average maximum second electrode surface remoteness is defined as a fourth distance d₄, wherein the fourth distance is the average of all distances between each point of a maximally distant curve and a closest surface of the second electrode at each point on the maximally distant curve. The maximally distant curve is entirely contained within the connected pathway and is maximally distant from all surfaces of the second electrode 152, 252.

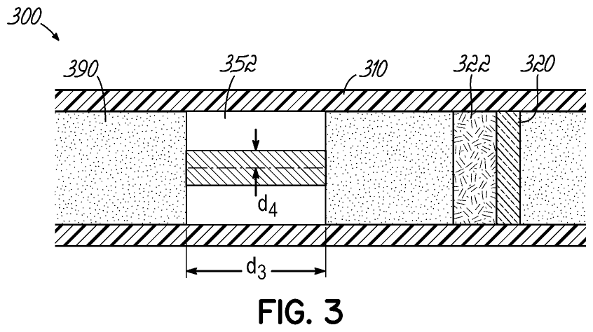

With regard to the above, FIG. 3 illustrated an example of such a permeable second electrode 352, which corresponds to the second electrode 152, 252 described above. In various embodiments of the disclosed invention, a device 300 includes at least one substrate 310 which can support, be exposed to, or transport a sample fluid 390. Device 300, substrate 310, and sample fluid 390 correspond respectively to device 100, 200, substrate 110, 210, and sample fluid 190, 290. The device 300 further includes at least one depleting component, which in FIG. 3 is represented by second electrode 352. In one such embodiment, second electrode 352 is an anode. In another such embodiment, second electrode 352 is a cathode. In various embodiments, the device 300 further includes at least one first electrode (not shown) which corresponds to first electrode 150, 250. In a further embodiment, the first electrode is a cathode. In a different further embodiment, the first electrode is an anode.

In this example, second electrode 352 includes a porous membrane with cylindrical pores that is coated with metal on all surfaces. The cylindrical pore is an example of a diffusion pathway, including a first end of the diffusion pathway on a first end of second electrode 352 and a second end of the diffusion pathway on a second end of second electrode 352. The first end of the diffusion pathway includes a point configured to allow entry of the at least one diffusant and the second end of the diffusion pathway includes a point configured to allow departure of the at least one diffusant which are in fluid communication with each other. Sample fluid 390, which corresponds to the sample fluid 190, 290 described above, the analyte, and the undesirable redox-active species it contains are examples of diffusants able to diffuse through the cylindrical pore of second electrode 352. In this example, the shortest diffusion pathway length, spanning a distance d₃, is equal to the length of the cylinder axis. In this example, the average maximum second electrode surface remoteness, denoted by a distance d₄, is calculated by averaging the distance between each point of a maximally distant curve, shown here as the axis of the cylinder, and a closest surface of the second electrode 352 at each point along the maximally distant curve, which is always one radius length away. Accordingly, in this example, the value of d₄ is equal to the radius of the cylindrical pore.

With further regard to FIG. 3, diffusants may interact with element 322 and/or third electrode 320, corresponding to element 122, 222 and third electrode 120, 220 described above respectively, after diffusing through electrode 352. In various embodiments, to aid in the reduction of undesirable redox-active species, a flow of sample fluid 390 is implemented where the sample fluid flows in the direction from first electrode 350 (not shown) toward the second electrode 352, element 322, and third electrode 320. This flow may be implemented by pumping, capillary, or other fluid flow inducing means.

In various embodiments of electrode 152, 252, 352, preferably d₃ is greater than d₄. In one such embodiment, preferably d₃ is at least two times greater than d₄. In a further embodiment, preferably d₃ is at least ten times greater than d₄. In a yet further embodiment, preferably d₃ is at least fifty times greater than d₄. In a further embodiment still, preferably d₃ is at least two hundred and fifty times greater than d₄. In an even further embodiment, preferably d₃ is at least one thousand times greater than d₄.

In various embodiments, the ratio between d₃ and d₄ is partially responsible for a significantly reduced concentration of undesirable redox-active species. In one such embodiment, the concentration of undesirable redox-active species is reduced to less than or equal to a third of the initial concentration. In a further embodiment, the concentration of undesirable redox-active species is reduced to less than or equal to a tenth of the initial concentration. In another further embodiment, the concentration of undesirable redox-active species is reduced to less than or equal to a thirtieth of the initial concentration. In a yet further embodiment, the concentration of undesirable redox-active species is reduced to less than or equal to a hundredth of the initial concentration. In another yet further embodiment, the concentration of undesirable redox-active species is reduced to less than or equal to a three hundredth of the initial concentration. In an even further embodiment, the concentration of undesirable redox-active species is reduced to less than or equal to a thousandth of the initial concentration.

Accordingly, an enzymatic sensor with a 10 μM analyte detection limit in interstitial fluid could use the present invention as disclosed above to achieve a significantly lower detection limit. In various embodiments, the original 10 μM analyte detection limit in interstitial fluid could be improved by implementing the present invention to achieve an analyte detection limit less than or equal to 1 μM. In one such embodiment, the original 10 μM analyte detection limit in interstitial fluid could be improved by implementing the present invention to achieve an analyte detection limit less than or equal to 10 nM.

### EXAMPLE

In one example, an electrochemical enzymatic glucose sensor was fabricated and evaluated in terms of a limit of detection for the at least one analyte under ideal sample fluid conditions, within a buffer and without the presence of undesirable redox-active species. The sensor was evaluated again, but with the addition of undesirable redox-active species, driving up the background signal not attributed to the target analyte and therefore increasing the limit of detection. To mitigate the effects of the undesirable redox-active species, a double-layer metal-coated porous membrane anode electrode was placed atop the enzymatic sensor and sealed, limiting the path of all outside diffusing species through the membrane electrode pores to the sensor. Two track-etch membranes were utilized, gold coated and facing each other, creating a tortuous path for diffusing species wherein the diffusion pathlength distance d₃ was at least one hundred times greater than the average maximum electrode surface distance d₄ between the diffusing species and the gold surface. Relative to a third gold wire electrode placed outside in a beaker containing the device and fluid, a constant fixed voltage was applied to the double-layer porous membrane electrode, oxidizing redox-active species which come in contact with the porous electrode, on its surface or within the pores. The enzymatic sensor with integrated double-layer porous membrane electrode, was evaluated again within a sample fluid containing undesirable redox-active species. The result was a decrease in the background signal and a decrease in limit of detection back to ideal sample fluid conditions as the undesirable redox-active species were electrochemically depleted by the porous membrane electrode before reaching the enzymatic sensor.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for detecting at least one analyte in a sample fluid, the device comprising:
a sensor for detecting an analyte-related redox-active species in a sample fluid; and
at least one depleting component that reduces the concentration of redox-active species in the sample fluid that are unrelated to the at least one analyte.

2. The device of claim 1, wherein the sensor further comprises an enzyme.

3. The device of claim 2, wherein the analyte-related redox-active species is produced by a reaction between the enzyme and the at least one analyte.

4. The device of claim 2, wherein the sensor further comprises an electrode.

5. The device of claim 4, wherein the analyte-related redox-active species is oxidized or reduced by the electrode.

6. The device of claim 1, wherein the analyte-related redox-active species is a redox-reporter that is bound to an aptamer.

7. The device of claim 6, wherein the sensor further comprises an electrode.

8. The device of claim 7, wherein the aptamer is bound to the electrode.

9. The device of claim 7, wherein the aptamer is free in solution and capable of interacting with the electrode.

10. The device of claim 7, wherein the electrode is an electrically-conductive diamond electrode.

11. The device of claim 1, wherein the sensor further comprises an element that contains or produces the analyte-related redox-active species.

12. The device of claim 11, wherein the element is an enzyme.

13. The device of claim 11, wherein the element is an aptamer.

14. The device of claim 13, further comprising a redox reporter associated with the aptamer.

15. The device of claim 1, wherein the at least one depleting component comprises at least one electrode.

16. The device of claim 15, wherein the at least one depleting component comprises a first electrode and a second electrode for depleting redox-active species in the sample fluid that are unrelated to the at least one analyte.

17. The device of claim 16, wherein the sensor further comprises a third electrode for detecting the analyte-related redox-active species.

18. The device of claim 17, wherein the first electrode and second electrode are separated by a first distance and the second electrode and third electrode are separated by a second distance, and the first electrode and third electrode are separated by a distance that is greater than the first distance and greater than the second distance.

19. The device of claim 18, wherein the first distance is greater than the second distance.

20. The device of claim 19, wherein the first distance is greater than the second distance by a factor chosen from at least 10×, at least 100×, and at least 1000×.

21. The device of claim 16, wherein the second electrode is configured to be permeable to at least one diffusant, wherein the at least one diffusant comprises:
the sample fluid;
the at least one analyte; and
redox-active species in the sample fluid that are unrelated to the at least one analyte.

22. The device of claim 21, wherein the second electrode is configured relative to the third electrode such that redox-active species in the sample fluid that are unrelated to the at least one analyte diffuse through the second electrode before interacting with the third electrode.

23. The device of claim 22, wherein redox-active species in the sample fluid that are unrelated to the at least one analyte diffuse through the second electrode before interacting with the sensor because the second electrode is placed on top of the sensor.

24. The device of claim 22, wherein the second electrode comprises:
a first end of the second electrode, wherein the first end of the second electrode comprises at least one point configured to allow entry of the at least one diffusant;
a second end of the second electrode, wherein the second end of the second electrode is located opposite of the first end of the second electrode and comprises at least one point configured to allow departure of the at least one diffusant; and
at least one diffusion pathway, wherein the diffusion pathway comprises:
at least one connected pathway, wherein the connected pathway comprises:
at least one first end of the connected pathway located on the first end of the second electrode, wherein the first end of the connected pathway comprises the point configured to allow entry of the at least one diffusant; and
at least one second end of the connected pathway located on the second end of the second electrode, wherein the second end of the connected pathway comprises the point configured to allow departure of the at least one diffusant, and wherein the point configured to allow entry of the at least one diffusant is in fluid communication with the point configured to allow departure of the at least one diffusant;
a diffusion pathway length, wherein the diffusion pathway length spans a first distance along a shortest path from the first end of the connected pathway to the second end of the connected pathway; and
an average maximum second electrode surface remoteness, wherein the average maximum second electrode surface remoteness comprises a second distance, wherein the second distance comprises an average of all distances between each points of a maximally distant curve and a closest surface of the electrode at each point along the maximally distant curve, wherein the maximally distant curve is entirely contained within the connected pathway and is maximally distant from all surfaces of the second electrode.

25. The device of claim 24, wherein the first distance is greater than the second distance.

26. The device of claim 25, wherein the first distance is greater than the second distance by a factor chosen from at least 2×, at least 10×, at least 50×, at least 250×, and at least 1000×.

27. The device of claim 25, wherein an initial concentration of redox-active species in the sample fluid that are unrelated to the at least one analyte can be significantly reduced in part because of the difference between the first distance and the second distance.

28. The device of claim 27, wherein the initial concentration of redox-active species in the sample fluid that are unrelated to the at least one analyte is reduced by a factor chosen from at least 3×, at least 10×, at least 30×, at least 100×, at least 300×, and at least 1000×.

29. The device of claim 25, wherein a limit of detection for the at least one analyte is improved by a factor chosen from at least 10× and at least 1000×.

30. The device of claim 17, wherein at least two of the electrodes are alternately connected to a voltage source.

31. The device of claim 1, wherein the at least one analyte has a concentration equal to or less than 1 mM or equal to or less than 1 μM.

32. A method for detecting at least one analyte in a sample fluid, the method comprising:
reducing the concentration of redox-active species in a sample fluid that are unrelated to the at least one analyte;
causing at least one analyte to interact with an element that:
(a) produces at least one analyte-related redox-active species in the presence of the at least one analyte;
(b) contains at least one analyte-related redox-active species which is made more detectable through interaction with the at least one analyte; or
(c) otherwise enables the detection of at least one analyte-related redox-active species attributable to the presence of the at least one analyte; and
detecting a presence or measuring an amount of the at least one analyte-related redox-active species attributable to the interaction between the at least one analyte and the element.

33. The method of claim 32, wherein the element comprises an enzyme.

34. The method of claim 32, wherein the element is an aptamer

35. The method of claim 34, further comprising a redox-reporter bound to the aptamer.

36. The method of claim 32, wherein causing the at least one analyte to interact with the element comprises causing the sample fluid to flow along a path, wherein that path comprises:
at least one depleting component that reduces the concentration of redox-active species in the sample fluid that are unrelated to the at least one analyte;
the element; and
an electrode.

37. The method of claim 36, wherein detecting a presence or measuring an amount of the analyte-related redox-active species further comprises measuring an initial electrical current between the at least one electrode and the at least one analyte-related redox-active species.

38. The method of claim 37, further comprising detecting and/or measuring a change from the initial electrical current between the at least one electrode and the at least one analyte-related redox-active species following bringing the sample fluid into proximity with the electrode.
